(19) United States
(12) Patent Application Publication
(10) Pub. No.: US 2021/0095393 A1
(43) Pub. Date: Apr. 1, 2021

(54) METHOD FOR PREPARING AMPLICON LIBRARY FOR DETECTING LOW-FREQUENCY MUTATION OF TARGET GENE

(71) Applicants: GENETRON HEALTH (BEIJING) CO, LTD., Beijing (CN); GENETRON HEALTH (BEIJING) LABORATORY CO., LTD., Beijing (CN); GENETRON HEALTH (CHONGQING) LABORATORY CO, LTD., Chongqing (CN)

(72) Inventors: Qiaosong ZHENG, Beijing (CN); Xiao SHI, Beijing (CN); Min CHEN, Beijing (CN); Kaihua ZHANG, Beijing (CN); Xiaoling GUO, Beijing (CN)

(21) Appl. No.: 16/757,222

(22) PCT Filed: Apr. 20, 2018
(86) PCT No.: PCT/CN2018/083822

§ 371 Date: Apr. 17, 2020

(30) Foreign Application Priority Data
Oct. 19, 2017 (CN) 201710976835.4

(51) Int. Cl. C40B 40/06; C12Q 1/6876; C12Q 1/6869

(52) U.S. Cl. CPC C40B 40/06; C12Q 1/6876; C12Q 2531/113; C12Q 2525/143; C12Q 1/6869

(57) ABSTRACT

The invention discloses a method for preparing an amplicon library for detecting a low-frequency mutation of a target gene. The invention provides a method for preparing an amplicon library for detecting a low-frequency mutation of a target gene, comprising the following steps: 1) design and synthesize a Barcode primer F1, an forward primer F2, a reverse outer primer R1, and a reverse inner primer R2; 2) perform a one-step PCR amplification on the cfDNA of the sample to be tested using the Barcode primer F1, the forward primer F2, the reverse outer primer R1, and the reverse inner primer R2 to obtain an amplified product, which is a DNA library for an amplicon sequencing. In addition to detect tissue samples, the method can also quickly, easily, sensitively and specifically amplify different target regions of cell free DNA from samples such as blood, urine, and CSF, and efficiently detect mutations as low as 0.1%. It greatly simplifies the experiment operation, effectively avoids library loss and contamination, significantly reduces costs and improves efficiency.

### RELATED APPLICATIONS

The present application is a National Phase of International Application Number PCT/CN2018/083822, filed Apr. 20, 2018, and claims the priority of Chinese Application No. 201710976835.4, filed Oct. 19, 2017.

### INCORPORATION BY REFERENCE

The sequence listing provided in the file entitled Sequence_Listing_2020-07-17_022w.txt, which is an ASCII text file that was created on Jul. 17, 2020, and which comprises 14,464 bytes, is hereby incorporated by reference in its entirety.

### TECHNICAL FIELD

The invention belongs to the field of biotechnology, and particularly relates to a method for preparing an amplicon library for detecting low-frequency mutations of a target gene.

### BACKGROUND OF THE INVENTION

Tumors are highly heterogeneous, and the pathogenic mutations may be present in extremely low proportions. The mutation site or mutation frequencies of the target gene in cfDNA in the blood, urine and cerebrospinal fluid (CSF) of a tumor patient will affect the judgment of administration of tumor drugs or tumor development direction in future. Therefore, detecting the mutation site or mutation region of the target gene in cfDNA in the blood, urine and CSF of a tumor patient has become the focus of research. This requires sequencing the mutation site or mutation region to detect the mutation frequencies.

At present, as the most accurate next-generation sequencing (NGS), Hiseq sequencing itself has an error rate of about 0.2%. In addition, the current DNA polymerase amplification error rate is also between 10⁻⁷-10⁻⁵. Thus, the key to directly obtain the low-frequency variation of the original template molecules in the sample is how to exclude amplification errors and sequencing errors in the sequencing results.

The content of cfDNA from blood, urine, and CSF of tumor patients is very low, which is a problem for the detection of low-frequency mutations. At present, there are three methods for detecting low-frequency mutations on the market: digital PCR, next-generation sequencing (NGS), and amplification refractory mutation system (ARMS) PCR. NGS has the advantages of high throughput, low cost, fast and simple operation. It is currently the most popular low-frequency mutation detection technology in China. For NGS, the first and most critical step in the entire sequencing process is to prepare a gene library. The quality of the gene library directly affects the subsequent sequencing work. However, conventional library preparing methods in the market all have the disadvantages of high cost, long detection cycle, complicated procedures, library is vulnerable to be contaminated and high requirements on the testing personnel, which are not suitable for preparing libraries of large sequencing samples.

### SUMMARY OF THE INVENTION

An object of the present invention is to provide a method for preparing an amplicon library for detecting mutation status in regions to be detected of target genes of a sample to be tested.

The method provided in the present invention is applicable to all NGS platforms and includes the following steps:

1) design and synthesize a Barcode primer F1, an forward primer F2, a reverse outer primer R1, and a reverse inner primer R2;

the Barcode primer F1 consists of a sequencing adapter 1, a barcode sequence for distinguishing different samples, and a common sequence 1 in this order;

the forward primer F2 consists of a common sequence 1, a molecular tag, a specific base sequence, and an forward specific primer sequence in this order;

the reverse outer primer R1 consists of a sequencing adapter 2 and a common sequence 2 in this order;

the reverse inner primer R2 consists of a common sequence 2 and a reverse specific primer sequence in this order;

the sequencing adapter 1 and the sequencing adapter 2 are corresponding sequencing adapters selected according to different sequencing platforms;

the barcode sequences are all nucleotides having a length of 8-12 nt, no continuous the same bases, and a GC content of 40-60%;

the common sequence 1 and the common sequence 2 have a length of 16-25 nt, no continuous the same bases, the GC content is 35-65%, and no obvious secondary structure;

the specific base sequence is GAT;

the forward specific primer sequence and the reverse specific primer sequence are primers for amplifying a region to be detected of the target gene;

the molecular tag is sequence having 10-12 random bases;

2) perform a one-step PCR amplification of the sample cfDNA to be tested using the Barcode primer F1, the forward primer F2, the reverse outer primer R1 and the reverse inner primer R2, to obtain an amplification product, which is a DNA library used for amplicon sequencing.

In the above method,

the sequencing platform is an Illumina platform, the sequencing adapter 1 is 15, and the sequencing adapter 2 is 17;

alternatively, the sequencing platform is an Ion Torrent platform, the sequencing adapter 1 is A, and the sequencing adapter 2 is P.

In the above method,

in the PCR amplification, a molar ratio of the Barcode primer F1, the forward primer F2, the reverse outer primer R1 and the reverse inner primer R2 is 6:(10-6):(1-3):(1-3).

In the above method,

the mutation is a low-frequency mutation, specifically, the mutation frequencies are as low as 0.1%.

In the above method,

the sample to be tested is cfDNA isolated from in vitro blood of a tumor patient, cfDNA isolated from in vitro urine of a tumor patient, cfDNA isolated from in vitro CSF of a tumor patient, or genomic DNA extracted from in vitro tumor tissues of a tumor patient.

A DNA library prepared by the method above is also within the protection scope of the present invention.

Use of the method above or the DNA library above in detecting mutation sites or mutation regions of target genes in the cfDNA of the sample to be tested is also within the protection scope of the present invention.

Use of the method above or the DNA library above in detecting mutation frequencies of mutation sites or mutation regions of target genes in the cfDNA of the sample to be tested is also within the protection scope of the present invention.

The detection of the mutation in the regions to be detected of the target genes of the sample to be tested is to detect mutant bases or amino acids of the regions to be detected in the target genes of the sample to be tested or to detect the mutation frequencies in the regions to be tested of the target genes of the sample to be tested.

The calculation method of mutation frequencies is as follows:

in the sequencing results, DNA molecules with the same molecular tag are amplification products of an original DNA template and are named as one family;

detect the mutation rate in the family, if the mutation rate of the family is 80%, the family is recorded as a mutant DNA family with a molecular tag;

mutation rate=(number of DNA molecules with mutations in codons encoding amino acid residues in the same family/total number of DNA molecules in the same family)*100%;

mutation frequencies=number of mutant DNA families with the molecular tag in the sequencing results/number of all DNA families with the molecular tag in the sequencing results*100%.

Note: when the number of reads (sequenced one sequence) with the same molecular tag in the sequencing result is ≥2, it is statistical significant.

Another object of the present invention is to provide a method for detecting a mutation status in a region to be detected of a target gene in cfDNA of a sample to be tested.

The method provided in the present invention includes the following steps:

1) prepare a DNA library according to the method of the first objective above;

2) sequence the DNA library to obtain a sequencing result, and analyze the mutation of the region to be tested of the target gene in cfDNA of the sample to be tested according to the sequencing results.

In the above method,

the nucleotide sequence of the common sequence 1 is SEQ ID No: 1 in the sequence listing;

the nucleotide sequence of the common sequence 2 is SEQ ID No: 2 in the sequence listing;

the nucleotide sequence of the sequencing adapter 1 is SEQ ID No: 3 in the sequence listing;

the nucleotide sequence of the sequencing adapter 2 is SEQ ID No: 4 in the sequence listing;

the barcode sequences for distinguishing different samples are SEQ ID Nos: 5 to 14 in the sequence listing, respectively;

the gene to be tested is NRAS, and the corresponding forward specific primer sequence and reverse specific primer sequence are SEQ ID NOs: 15 and 16 or SEQ ID NOs: 17 and 18 in the sequence listing, respectively;

the gene to be tested is ALK, and the corresponding forward specific primer sequence and reverse specific primer sequence are SEQ ID NOs: 19 and 20 or SEQ ID NOs: 21 and 22 or SEQ ID NOs: 23 and 24 or SEQ ID NOs: 25 and 26 or SEQ ID NOs: 27 and 28 or SEQ ID NOs: 29 and 30 or SEQ ID NOs: 31 and 32 in the sequence listing, respectively;

the gene to be tested is PIK3CA, and the corresponding forward specific primer sequence and reverse specific primer sequence are SEQ ID NOs: 33 and 34 or SEQ ID No: 35 or SEQ ID No: 36 in the sequence listing, respectively;

the gene to be tested is ROS, and the corresponding forward specific primer sequence and reverse specific primer sequence are SEQ ID NOs: 37 and 38 in the sequence listing, respectively;

the gene to be tested is EGFR, and the corresponding forward specific primer sequence and reverse specific primer sequence are SEQ ID NOs: 39 and 40 or SEQ ID NOs: 41 and 42 or SEQ ID NOs: 43 and 44 or SEQ ID NOs: 45 and 46 or SEQ ID NOs: 47 and 48 in the sequence listing, respectively;

the gene to be tested is MET, and the corresponding forward specific primer sequence and reverse specific primer sequence are SEQ ID NOs: 49 and 50 or SEQ ID NOs: 51 and 52 and SEQ ID NOs: 53 and 54 or SEQ ID NOs: 55 and 56 in the sequence listing, respectively;

the gene to be tested is BRAF, and the corresponding forward specific primer sequence and reverse specific primer sequence are SEQ ID NOs: 57 and 58 or SEQ ID NOs: 59 and 60 in the sequence listing, respectively;

the gene to be tested is KRAS, and the corresponding forward specific primer sequence and reverse specific primer sequence are SEQ ID NOs: 61 and 62 or SEQ ID NOs: 63 and 64 in the sequence listing, respectively;

the gene to be tested is TP53, and the corresponding forward specific primer sequence and reverse specific primer sequence are SEQ ID NOs: 65 and 66 or SEQ ID NOs: 67 and 68 or SEQ ID NOs: 69 and 70 or SEQ ID NOs: 71 and 72 or SEQ ID NOs: 73 and 74 or SEQ ID NOs: 75 and 76 in the sequence listing, respectively;

the gene to be tested is ERBB2, and the corresponding forward specific primer sequence and reverse specific primer sequence are SEQ ID NOs: 77 and 78 in the sequence listing, respectively.

The sample to be tested above is cfDNA isolated from in vitro blood of a tumor patient, cfDNA isolated from in vitro urine of a tumor patient, cfDNA isolated from in vitro CSF of a tumor patient, or genomic DNA extracted from in vitro tumor tissues of a tumor patient.

A third object of the present invention is to provide a method for detecting mutation frequencies of mutation sites or mutation regions of target genes in a sample to be tested.

The method provided in the present invention comprises the steps of the above method, wherein the sample to be tested is cfDNA isolated from in vitro blood of a tumor patient, cfDNA isolated from in vitro urine of a tumor patient, and cfDNA isolated from in vitro CSF of a tumor patient, or genomic DNA extracted from tumor tissue of a tumor patient.

The above method or the above-mentioned DNA library is used in guiding administration of tumor drugs or judging tumor development direction.

A fourth object of the present invention is to provide a method for guiding administration of tumor drugs for a patient to be tested or judging a tumor development direction.

A method is provided in the present invention, comprising firstly detect the mutation frequencies of mutation sites or mutation regions of target genes in cfDNA of a patient to be tested using the steps of methods described above, then guide the administration of tumor drugs or judge the tumor development direction of the patient to be tested according to the mutation frequencies.

A fifth object of the present invention is to provide a kit for an amplicon library for detecting mutation status in regions to be detected of target genes of a sample to be tested.

The kit provided in the present invention includes a Barcode primer F1, an forward primer F2, a reverse outer primer R1, and a reverse inner primer R2 mentioned in the above method.

### BRIEF DESCRIPTION OF THE DRAWINGS

FIG. 1 shows use of molecular tags. A, B and C represent different mutation sites, respectively.

FIG. 2 shows a distribution of amplified products detected by Agilent 2200 TapeStation Systems after the preparation of a library for cfDNA extracted from the blood sample of subject 1.

FIG. 3 shows the sequencing result on the Ion Torrent platform of the amplicon library obtained from the one-step method for cfDNA of the blood sample of subject 1.

### BEST MODE OF IMPLEMENTING THE INVENTION

Unless otherwise specified, the experimental methods used in the following examples are conventional methods.

Unless otherwise specified, the materials, reagents in the following examples are commercially available.

### Example 1. Preparation of an Amplicon Library for Detecting Low-Frequency Mutations of a Target Gene

The mutation frequencies of mutation sites or mutation regions of target genes in cfDNA from the blood, urine and CSF of tumor patients will affect the judgment of administration of tumor drugs or tumor development direction in the future. In this example, in order to detect mutation frequencies of mutation sites or mutation regions of target genes in cfDNA from the blood, urine and CSF of a tumor patient, an amplicon library for detecting low-frequency mutations of the target gene is prepared as follows:

I. Design and Synthesize of Primer Combinations for Amplicon Libraries for Detecting Low-Frequency Mutations of the Target Gene

Select a region of a known target gene as the region to be tested, and design and synthesize the following primers:

there are mutation hotspots in this region to be tested, but whether the gene mutation in the sample to be tested is known or unknown

Barcode primer F1: sequencing adapter 1+barcode sequence+common sequence 1;

Forward primer F2: common sequence 1+molecular tag+specific base sequence+forward specific primer sequence;

Reverse outer primer R1: sequencing adapter 2+common sequence 2;

Reverse internal primer R2: common sequence 2+reverse specific primer sequence;

wherein, the barcode sequence is a sequence for distinguishing different samples. One sample to be tested corresponds to one barcode sequence. The length of the barcode sequence is 8-12 nt. It is required that there are no continuous the same bases, and the GC content is 40-60%. The primers into which the barcode sequence is introduced have no obvious secondary structure. F1 is used to distinguish different samples. As long as the samples are the same, F1 is the same regardless of the detection site.

The length of common sequences 1 and 2 is 16-25 nt. It is required that there are no continuous the same bases, and the GC content is 35-65%. The primers into which the barcode sequence is introduced have no obvious secondary structure. The sequence can be changed as needed. Those used in this example are as follows:

Common sequence 1
(SEQ ID NO: 1)
GGCATACGTCCTCGTCTA,
18nt in size;
Common sequence 2
(SEQ ID NO: 2)
CGACATCGCCTCTGCTGT,
18nt in size.

Sequencing adapter 1 and sequencing adapter 2 are determined according to the sequencing platform:

If the sequencing platform is an Illumina platform, sequencing adapters 1 and 2 are 15 and 17, respectively. The adapter sequence and the primer sequence on the chip are complementary. The added adapter is used to connect the nucleic acid fragment to the vector.

If the sequencing platform is an Ion Torrent platform, sequencing adapters 1 and 2 are A and P (SEQ ID NOs: 3 and 4), respectively. The adapter A is used for sequencing and is complementary to the specific primers. The adapter P is complementary to the sequence on the vector and used to connect the template to the vector.

The specific base sequence is GAT, which is not a part of the gene specific amplified fragment. It is used to facilitate the analysis of biological information of sequencing results and improve the efficiency of data screening by identifying GAT sequences.

The forward specific primer sequence and the reverse specific primer sequence are primers designed to be used to amplify the region to be tested of a target gene. The forward specific primer sequence has a size of 15-30 nt and the reverse specific primer sequence has a size of 15-30 nt.

The molecular tag is a sequence having 10-12 random bases, used to mark the original cfDNA template. Each random base has four base forms of ATCG, so there are a total of 1048576 different molecular tags for the 10 random bases. Taking the original 20 ng DNA template as an example, its copy number is 6000, and the molecular fragment of cfDNA is shorter, so the effective template copy number that can be amplified is less than 6000, and 1048576 molecular tags can be used to add a specific “tag” to each original template. Classification of the original template of sequencing results by molecular tags can eliminate amplification errors and sequencing errors.

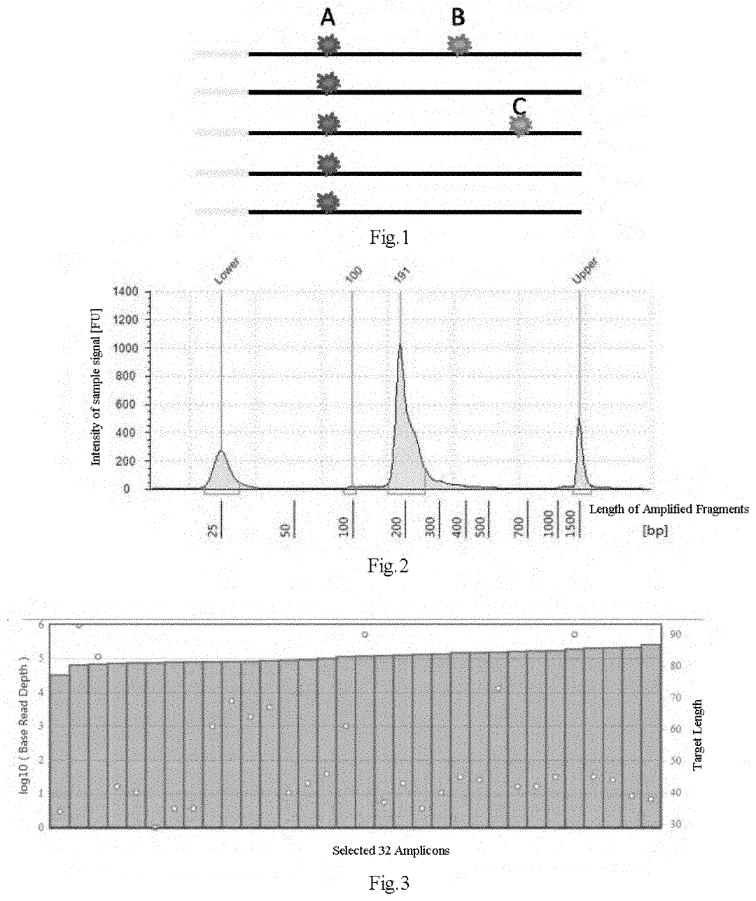

As shown in FIG. 1, FIG. 1 shows the five amplified products with the same molecular tag in the prepared library, in which the mutation at the site A is present on all five molecules, and the mutation at the site B or the mutation at the site C is just present in one of the amplification products, and the proportion is extremely low. Thus, it can be determined that the mutation at the site A is the mutation present in the original template molecule, and the mutation at the site B or the mutation at the site C is false positive mutation that occurs during the PCR amplification for the preparation of the library or during sequencing. Therefore, the use of the molecular tag is to mark the original template molecule, identify the mutations present in the original template, eliminate false positive mutations during PCR and sequencing, and improve detection sensitivity.

II. The Establishment of Detection Method

1. The primers F2, R1, and R2 mentioned in above I are mixed according to a specific ratio, then named as the Primer Mix after thoroughly mixing and being ready for use.

2. Extracting cfDNA from samples to be tested, such as the blood, urine or CSF of a tumor patient.

3. PCR amplification was performed on the cfDNA with barcode primers and primer mix corresponding to different samples, and the reagents shown in Table 1 below were sequentially added to 8-strip tubes of 0.2 ml or 96-well plates to obtain a PCR amplification system.

Table 1 shows the PCR amnlification svslem

| PCR component | Dosage | |
|---|---|---|
| KAPA HiFi HotStart ReadyMix | 15 | μl |
| (KAPA KK2602) | | |
| Template DNA | 5-50 | ng |
| Barcode primers (50 μM) | 1 | μl |
| Primer Mix (50 μM) | 2 | μl |

| | |
|---|---|
| DNAase-free H₂O | adding water to |
| | 30 μl |

Among them, in the primer mix, R1, F2, and R2 primers were added at an original concentration of 50 μM, with a volume ratio of R1:F2:R2=10:(1-5):(1-5).

In the PCR amplification system, the molar ratio of Barcode primer F1, forward primer F2, reverse outer primer R1 and reverse inner primer R2 is as follows: the molar ratio of F1:R1:F2:R2=6:(10-6):(1-3):(1-3)

4. The amplification procedure shown in Table 2 below is performed with a PCR instrument (the PCR instrument used is Bio-System 2720 Thermal Cycler):

Table 2 shows the amplification procedure

| Temperature | Time | | Number of cycles |
|---|---|---|---|
| 95° C. | 3 | m | 1 Cycle |
| 95° C. | 30 | s | 2 Cycles |
| 64° C. | 30 | | |
| 62° C. | 30 | | |
| 60° C. | 30 | | |
| 58° C. | 30 | s | |
| 72° C. | 90 | s | |
| 72° C. | 5 | m | 1 Cycle |
| 95° C. | 3 | min | 1 Cycle |
| 95° C. | 30 | s | 19 Cycles |
| 60° C. | 90 | s | |
| 72° C. | 90 | s | |
| 72° C. | 10 | m | 1 Cycle |

The cycle conditions of the gradient annealing temperature in the first two cycles of the library preparing PCR process are preliminary amplification of the original template. That is, specific molecular tags are added to different original templates. Then, the subsequent 19 cycles of PCR are used for amplification of the original template inside the molecular tags. At the same time, the high concentration of F1, R1 and the low concentration of primers F2, R2 also ensure that the amplification inside molecular tags is performed during the following 19 cycles (that is, generally, other molecular tags are not added during the amplification).

5. 1.3 times of the Agencourt AMPure XP Kit (BECKMAN COULTER, A63882) of the PCR reaction solution is drawn by pipette to purify and recover the PCR product to obtain a DNA library for amplicon sequencing. The specific purification steps are as follows:

1) take out the Agencourt AMPure XP Kit 30 minutes in advance, vortex thoroughly, and let it stand at room temperature.

2) after the PCR reaction is completed, vortex the magnetic beads thoroughly again, add 24 μl of magnetic beads to the system, blow and beat repeatedly more than 5 times or vortex thoroughly, and let it stand at room temperature for 5 minutes.

3) transfer the EP tube to a magnetic stand, and let it stand for 5 minutes until the solution is clear. The supernatant is carefully removed with a pipette, without touching the magnetic beads.

4) add 100 μl of freshly prepared 80% of ethanol solution to each tube, placing the EP tube on a magnetic stand and rotating it slowly 2 times, letting it stand for 5 minutes, and discarding the supernatant.

5) repeat step 4) once.

6) open the EP tube and letting it stand at room temperature to make the liquid volatilize completely and the surface of the magnetic beads is dim. Take care not to dry the magnetic beads excessively.

7) Take out the EP tube from the magnetic stand, add 30 μl of PCR-grade purified water, mix by vortexing, and let it stand at room temperature for 10 minutes.

8) Place the EP tube from the previous step on a magnetic stand for 2 minutes or until the solution is clear. Carefully suck the supernatant on the side away from the magnet with pipette, without touching the magnetic beads.

Now, the preparation of the amplicon library is completed, and QuBit quantification is used to determine whether the preparation is successful.

6. Computer sequencing and analysis of results

The libraries amplified from the different samples are mixed in equal proportions according to the determined concentration, and finally diluted to a specific concentration, and then sequenced with a next-generation sequencer to obtain sequencing results.

The mutation status of the detected genes is obtained after the results of sequencing were analyzed for data processing and bioinformatics analysis. The data processing includes transformation of sequencing data, quality control, sequence alignment (reference genome is NCBI GRCh37/Hg19), mutation site analysis. After data processing and analysis, the mutation status and mutation frequencies of the sample to be tested are obtained.

Since the original template is molecularly labeled during the library amplification, the mutation frequencies are calculated as follows:

In the sequencing results, the DNA molecule with the same molecular tag is an amplification product of an original DNA template and is named as one family;

Detect the mutation rate in the family. If the mutation rate of the family is ≥80%, the family is recorded as a mutant DNA family with a molecular tag;

Mutation rate=(number of DNA molecules with mutations in codons encoding amino acid residues in the same family/total number of DNA molecules in the same family)*100%;

Mutation frequencies=number of mutant DNA families with molecular tags in the sequencing results/number of all DNA families with molecular tags in the sequencing results*100%.

Note: when the number of reads (sequence sequenced) with the same molecular tag in the sequencing result is ≥2, it is statistical significant.

### Example 3 Preparation of an Amplicon Library for Detecting Low-Frequency Mutations of a Target Gene

The target genes are shown in Table 5. The samples to be tested are derived from 49 patients who have been identified as patients with lung cancer. The purpose of this example is to detect the mutation frequencies of the 49 patients shown in Table 5 by the method of the present invention.

I. Design and Synthesis of Primer Combinations for Amplicon Libraries for Detecting Low-Frequency Mutations of Target Genes

The following primers were designed and synthesized according to the mutation sites or the mutation regions of the target gene in I of Example 1, as shown in Tables 3 and 4:
Table 3 shows primer combinations

| | | |
|---|---|---|
| Barcode | Sequencing | CCATCTCATCCCTGCGTGTCTCCGACTCAG |
| primers | adapter 1 | (SEQ ID NO: 3) |
| (F1) | Barcode | TCCTCGAATC(SEQ ID NO: 5) |
| | sequence | TAGGTGGTTC(SEQ ID NO: 6) |
| | | TCTAACGGAC(SEQ ID NO: 7) |
| | | TTGGAGTGTC(SEQ ID NO: 8) |
| | | TCTAGAGGTC(SEQ ID NO: 9) |
| | | TCTGGATGAC(SEQ ID NO: 10) |
| | | TCTATTCGTC(SEQ ID NO: 11) |
| | | AGGCAATTGC(SEQ ID NO: 12) |
| | | TTAGTCGGAC(SEQ ID NO: 13) |
| | | CAGATCCATC(SEQ ID NO: 14) |
| | Common | GGCATACGTCCTCGTCTA |
| | sequence 1 | (SEQ ID NO: 1) |
| Forward | Common | GGCATACGTCCTCGTCTA |
| primers | sequence 1 | (SEQ ID NO: 1) |
| (F2) | Molecular tag | NNNNNNNNNN(SEQ ID NO: 79) |
| | Specific base | GAT |
| | sequence | |
| | Forward | Corresponding sequence of the |
| | specific | forward specific primers |
| | primers | of each gene shown in Table 4 |
| | sequence | |
| Reverse | Sequencing | CCTCTCTATGGGCAGTCGGTGAT |
| outer | adapter 2 | (SEQ ID NO: 4) |
| primers | Common | CGACATCGCCTCTGCTGT |
| (R1) | sequence 2 | (SEQ ID NO: 2) |
| Reverse | Common | CGACATCGCCTCTGCTGT |
| inner | sequence 2 | (SEQ ID NO: 2) |
| primers | Sequence of | Corresponding sequence of the |
| (R2) | the reverse | reverse specific primers of |
| | specific | each gene shown in Table 4 |
| | primers | |

The principles for specific primer design: annealing temperature is 55-65° C., as few secondary structures as possible, GC content is 35%-65%, primer length is 16-30 nt, secondary structures should not be formed between primers, as shown in Table 4.
Table 4 shows the primer combinations of specific primer sequences corresponding to each gene.

| | Gene | Sequence of the | Sequence of the |
|---|---|---|---|
| No. | name | forward specific primers | reverse specific primers |
| 1 | NRAS-1 | GGTGAAACCTGTTTGTTGGACAT | CTTCGCCTGTCCTCATGTATTG |
| | | (SEQ ID NO: 15) | (SEQ ID NO: 16) |
| 2 | NRAS-2 | TGGTGTGAAATGACTGAGTACAAACTG | GTTCTGGATTAGCTGGATTGTCAGT |
| | | (SEQ ID NO: 17) | (SEQ ID NO: 18) |
| 3 | ALK-1 | TCCAGGCCCTGGAAGAGT | TGAGGCAGTCTTTACTCACCTGTAG |
| | | (SEQ ID NO: 19) | (SEQ ID NO: 20) |
| 4 | ALK-2 | CCTGTGGCTGTCAGTATTTGGAG | ACACAGATCAGCGACAGGATG |
| | | (SEQ ID NO: 21) | (SEQ ID NO: 22) |
| 5 | ALK-3 | AATCCCTGCCCCGGTT | GGGCGGGTCTCTCGG |
| | | (SEQ ID NO: 23) | (SEQ ID NO: 24) |
| 6 | ALK-4 | GTTAATTTTGGTTACATCCCTCTCTGC | GATTGCAGGCTCACCCCAA |
| | | (SEQ ID NO: 25) | (SEQ ID NO: 26) |
| 7 | ALK-5 | ACTGGATTTCCTCATGGAAGCC | AGATATCGATCTGTTAGAAA |
| | | (SEQ ID NO: 27) | CCTCTCCA(SEQ ID NO: 28) |
| 8 | ALK-6 | CGGACTCTGTAGGCTGCAGT | GGAAATCCAGTTCGTCCTGTTCA |
| | | (SEQ ID NO: 29) | (SEQ ID NO: 30) |
| 9 | ALK-7 | GTTTGACTCTGTCTCCTCTTGTCTTC | CTTGGGTCGTTGGGCATTC |
| | | (SEQ ID NO: 31) | (SEQ ID NO: 32) |
| 10 | PIK3CA-1 | CAAAGAACAGCTCAAAGCAATTTCTAC | ATTTTAGCACTTACCTGTGACTCCATAG |
| | | (SEQ ID NO: 33) | (SEQ ID NO: 34) |
| 11 | PIK3CA-2 | AGCAAGAGGCTTTGGAGTATTTCAT | TGTGTGGAAGATCCAATCCATTTTTG |
| | | (SEQ ID NO: 35) | (SEQ ID NO: 36) |
| 12 | ROS1 | CTTCCCTCGGGAAAAACTGAC | GATGTCCACTGCTGTTCCTTCAT |
| | | (SEQ ID NO: 37) | (SEQ ID NO: 38) |
| 13 | EGFR-1 | CCAACCAAGCTCTCTTGAGGAT | CACCGTGCCGAACGC |
| | | (SEQ ID NO: 39) | (SEQ ID NO: 40) |
| 14 | EGFR-2 | CCCAGAAGGTGAGAAAGTTAAAATTCC | CACATCGAGGATTTCCTTGTTGG |
| | | (SEQ ID NO: 41) | (SEQ ID NO: 42) |
| 15 | EGFR-3 | CTCTCCCTCCCTCCAGGA | GAGGCAGATGCCCAGCA |
| | | (SEQ ID NO: 43) | (SEQ ID NO: 44) |
| 16 | EGFR-4 | CTGCCTCACCTCCACCG | ATTGTCTTTGTGTTCCCGGACA |
| | | (SEQ ID NO: 45) | (SEQ ID NO: 46) |
| 17 | EGFR-5 | GGAGGACCGTCGCTTGGCTTCC | CTTCTGCATGGTATTCTTTCT |
| | | (SEQ ID NO: 47) | (SEQ ID NO: 48) |
| 18 | MET-1 | CTTGTAAGTGCCCGAAGTGTAAG | GTCACAACCCACTGAGGTATATGT |
| | | (SEQ ID NO: 49) | (SEQ ID NO: 50) |
| 19 | MET-2 | CTAACCAAGTTCTTTCTTTTGCACAG | AGCACAGTGAATTTTCTTGCCATC |
| | | (SEQ ID NO: 51) | (SEQ ID NO: 52) |
| 20 | MET-3 | CAGTCAAGGTTGCTGATTTTGGT | CTTTGCACCTGTTTTGTTGTGTAC |
| | | (SEQ ID NO: 53) | (SEQ ID NO: 54) |
| 21 | MET-4 | GGTGCAAAGCTGCCAGTG | AACCAATACATTACCACATCTGACTTG |
| | | (SEQ ID NO: 55) | (SEQ ID NO: 56) |
| 22 | BRAF-1 | CTTCATGAAGACCTCACAGTAAAAATAGG | CTCAATTCTTACCATCCACAAAATGG |
| | | (SEQ ID NO: 57) | (SEQ ID NO: 58) |
| 23 | BRAF-2 | GGGCAGATTACAGTGGGACA | AATGTCACCACATTACATACTTACCATG |
| | | (SEQ ID NO: 59) | (SEQ ID NO: 60) |
| 24 | KRAS-1 | GGAGAAACCTGTCTCTTGGATATTCTC | TCCTCATGTACTGGTCCCTCAT |
| | | (SEQ ID NO: 61) | (SEQ ID NO: 62) |
| 25 | KRAS-2 | AGGCCTGCTGAAAATGACTGA | GAATTAGCTGTATCGTCAAGGCACT |
| | | (SEQ ID NO: 63) | (SEQ ID NO: 64) |
| 26 | TP53-1 | GTGTATATACTTACTTCTCCCCCTCCT | CCTCATTCAGCTCTCGGAACAT |
| | | (SEQ ID NO: 65) | (SEQ ID NO: 66) |
| 27 | TP53-2 | CCTATCCTGAGTAGTGGTAATCTACTGG | CCCTTTCTTGCGGAGATTCTC |
| | | (SEQ ID NO: 67) | (SEQ ID NO: 68) |
| 28 | TP53-3 | TCTCCTAGGTTGGCTCTGACTGTA | CCTGGAGTCTTCCAGTGTGAT |
| | | (SEQ ID NO: 69) | (SEQ ID NO: 70) |
| 29 | TP53-4 | GCATCTTATCCGAGTGGAAGGAAAT | CCTCCCAGAGACCCCAGT |
| | | (SEQ ID NO: 71) | (SEQ ID NO: 72) |
| 30 | TP53-5 | CTGTGGGTTGATTCCACACC | CTCACCATCGCTATCTGAGCA |
| | | (SEQ ID NO: 73) | (SEQ ID NO: 74) |
| 31 | TP53-6 | GCATTCTGGGACAGCCAAG | TACGGCCAGGCATTGAAGTA |
| | | (SEQ ID NO: 75) | (SEQ ID NO: 76) |
| 32 | ERBB2 | TCCCATACCCTCTCAGCGT | CCAGAAGGCGGGAGACATATG |
| | | (SEQ ID NO: 77) | (SEQ ID NO: 78) |

II. Detection

1. As 1 of II in Example 1, mix the three primers R1, F2, and R2 according to a specific ratio, and after thoroughly mixing, they are called the Primer Mix. The molar ratio of F1:R1:F2:R2=6:10:1:1, and being ready for use.

The concentration of Barcode primer F1 is 1.67 μM;

the concentration of the reverse outer primer R1 is 2.78 μM;

the concentration of the forward primer F2 is 0.28 μM;

the concentration of the reverse inner primer R2 is 0.28 μM.

2. The FFPE samples (paraffin-embedded tissues after formalin fixation) and blood samples were collected from 49 subjects (all of whom have been diagnosed with cancer), and the genomic DNA of FFPE samples and blood samples cfDNA are extracted.

3. It is the same as 3 of II in Example 1.

4. It is the same as 4 of II in Example 1.

5. It is the same as 5 of II in Example 1;

the detection results of the library of subject 1 (comprising 32 amplicons) are shown in FIG. 2. FIG. 2 shows the distribution map of the amplified products detected by Agilent 2200 TapeStation Systems after the library is prepared. The horizontal coordinate represents the length of the fragment. The vertical coordinate represents the signal intensity (FU), and the lower peak shows a marker at 25 bp position, and the upper peak shows a marker at 1500 bp position. As shown in FIG. 2, the PCR products obtained after PCR amplification are concentrated in the range of 160-230 bp.

6. Computer sequencing and the result analysis are the same as those in 5 of II of Example 1. The results are as follows:

FIG. 3 shows the sequencing results of cfDNA extracted from a blood sample (sample 1) of a patient diagnosed with lung cancer using the present library preparing method of the Ion Torrent platform.

The test results of DNA from FFPE samples and cfDNA from blood samples corresponding to the 49 subjects are shown in Table 5 below. The comparison methods of FFPE DNA and blood sample cfDNA tests were performed by Agilent's SureSelect customized service to capture and prepare the library. As for the detection method II of cfDNA from blood samples, the method of this patent is used to prepare a library. The results show that the consistency between genomic DNA from FFPE samples and cfDNA from blood samples detected by this patent method is as high as 87.76%. This patent method and Agilent SureSlect custom service are used at the same time to capture and prepare the library for cfDNA of 49 subjects, and the consistency is as high as 95.92%. As for 2 inconsistent site mutations, it has been verified by ddPCR that the results of the mutation detection obtained by the method of this patent are consistent with that obtained in ddPCR. It proves that the detection sensitivity of cfDNA in the present method is superior than the detection of cfDNA by capturing method. All these fully illustrate the practical applicability and good specificity of the present invention.

Table 5 shows the test results of DNA from FFPE samples and cfDNA from blood samples corresponding to 49 subjects.

| | | | detection of DNA | Detection of | | |
|---|---|---|---|---|---|---|
| | | | from FFPE by | cfDNA from | Detection of cfDNA | |
| | Information | | capturing | blood by the | from blood by | |
| | on mutations | | method | present method | capturing method | |
| sample | Genes | Protein_Change | mutation rate | mutation rate | mutation rate | Note |
| sample 1 | | | No hotspot | No hotspot | No hotspot | |
| | | | mutation | mutation | mutation | |
| sample 2 | | | No hotspot | No hotspot | No hotspot | |
| | | | mutation | mutation | mutation | |
| sample 3 | EGFR | p.L858R | 13% | no mutation | no mutation | Mutation is present in |
| | | | | detected | detected | tissues; but no mutation |
| | | | | | | is detected with both |
| | | | | | | methods for cfDNA |
| sample 4 | EGFR | p.L858R | 21.50% | 12.70% | 11.74% | |
| | TP53 | p.R273L | 17.41% | 15.39% | 15.22% | |
| | EGFR | p.T790M | 5.40% | 2.15% | 2.37% | |
| sample 5 | | | No hotspot | No hotspot | No hotspot | |
| | | | mutation | mutation | mutation | |
| sample 6 | | | No hotspot | No hotspot | No hotspot | |
| | | | mutation | mutation | mutation | |
| sample 7 | KRAS | p.G12V | 21.46% | 6.03% | 6.21% | |
| sample 8 | | | No hotspot | No hotspot | No hotspot | |
| | | | mutation | mutation | mutation | |
| sample 9 | | | No hotspot | No hotspot | No hotspot | |
| | | | mutation | mutation | mutation | |
| sample 10 | | | No hotspot | No hotspot | No hotspot | |
| | | | mutation | mutation | mutation | |
| sample 11 | EGFR | p.E746_A7 | 14.90% | 2.64% | 2.59% | |
| | | 50del | | | | |
| sample 12 | | | No hotspot | No hotspot | No hotspot | |
| | | | mutation | mutation | mutation | |
| sample 13 | | | No hotspot | No hotspot | No hotspot | |
| | | | mutation | mutation | mutation | |
| sample 14 | | | No hotspot | No hotspot | No hotspot | |
| | | | mutation | mutation | mutation | |
| sample 15 | EGFR | p.L858R | 6.80% | 8.06% | 7.63% | |
| sample 16 | TP53 | p.R248W | 7.83% | 0.40% | 0.42% | |
| | TP53 | p.C176F | No hotspot | 0.15% | 0.19% | No mutation is present |
| | | | mutation | | | in tissues; but mutation |
| | | | | | | is detected with both |
| | | | | | | methods for cfDNA |
| sample 17 | EGFR | p.L858R | 5.50% | 5.02% | 4.97% | |
| sample 18 | | | No hotspot | No hotspot | No hotspot | |
| | | | mutation | mutation | mutation | |
| sample 19 | KRAS | p.G12C | 11.20% | No hotspot | No hotspot | Mutation is present in |
| | | | | mutation | mutation | tissues; but no mutation |
| | | | | | | is detected with both |
| | | | | | | methods for cfDNA |
| sample 20 | EGFR | p.A767_V7 | 26.40% | 2.74% | 2.49% | |
| | | 69dup | | | | |
| sample 21 | PIK3 | p.E545K | 18.40% | 0.47% | 0.52% | |
| | CA | | | | | |
| sample 22 | TP53 | p.V157F | 7.20% | 1.36% | 1.38% | |
| sample 23 | EGFR | p.G719S | 72.90% | 2.99% | 2.84% | |
| | EGFR | p.E709A | 73.00% | 2.88% | 2.81% | |
| | ALK | p.F1245V | 26.90% | 0.62% | 0.59% | |
| sample 24 | EGFR | p.E746_A7 | 17.12% | 0.28% | No hotspot | verification by digital |
| | | 50del | | | mutation | PCR is positive |
| sample 25 | | | No hotspot | No hotspot | No hotspot | |
| | | | mutation | mutation | mutation | |
| sample 26 | TP53 | p.A273C | 67.58% | 4.69% | 4.66% | |
| | TP53 | p.R249S | 45.70% | 2.03% | 2.12% | |
| sample 27 | | | No hotspot | No hotspot | No hotspot | |
| | | | mutation | mutation | mutation | |
| sample 28 | TP53 | p.Y220C | 6.42% | 4.27% | 4.51% | |
| sample 29 | EGFR | p.E746_A7 | 10.20% | 7.71% | 8.37% | |
| | | 50del | | | | |
| sample 30 | EGFR | p.L747_T7 | 16.20% | 1.91% | 1.73% | |
| | | 51del | | | | |
| | TP53 | p.R337L | 11.50% | No hotspot | No hotspot | Mutation is present in |
| | | | | mutation | mutation | tissues; but no mutation |
| | | | | | | is detected with both |
| | | | | | | methods for cfDNA |
| sample 31 | | | No hotspot | No hotspot | No hotspot | |
| | | | mutation | mutation | mutation | |
| sample 32 | | | No hotspot | No hotspot | No hotspot | |
| | | | mutation | mutation | mutation | |
| sample 33 | TP53 | p.H168P | 25.80% | 0.45% | 0.46% | |
| sample 34 | | | No hotspot | No hotspot | No hotspot | |
| | | | mutation | mutation | mutation | |
| sample 35 | EGFR | p.L858R | 37.40% | 0.13% | No hotspot | verification by digital |
| | | | | | mutation | PCR is positive |
| | TP53 | p.C176Y | 78.80% | 0.87% | 0.94% | |
| sample 36 | | | No hotspot | No hotspot | No hotspot | |
| | | | mutation | mutation | mutation | |
| sample 37 | TP53 | p.Y220C | 80.70% | 0.73% | 0.63% | |
| | EGFR | p.L747_P7 | 65.50% | 1.39% | 1.26% | |
| | | 53delinsS | | | | |
| sample 38 | | | No hotspot | No hotspot | No hotspot | |
| | | | mutation | mutation | mutation | |
| sample 39 | | | No hotspot | No hotspot | No hotspot | |
| | | | mutation | mutation | mutation | |
| sample 40 | TP53 | p.H179A | 68.50% | 7.74% | 7.48% | |
| | PIK3 | p.E542K | 43.00% | 6.03% | 5.93% | |
| | CA | | | | | |
| sample 41 | | | No hotspot | No hotspot | No hotspot | |
| | | | mutation | mutation | mutation | |
| sample 42 | | | No hotspot | No hotspot | No hotspot | |
| | | | mutation | mutation | mutation | |
| sample 43 | TP53 | p.A248Y | 24.30% | 0.50% | 0.48% | |
| | EGFR | p.E746_T7 | 18.20% | No hotspot | No hotspot | Mutation is present in |
| | | 51delinsA | | mutation | mutation | tissues; but no mutation |
| | | | | | | is detected with both |
| | | | | | | methods for cfDNA |
| sample 44 | | | No hotspot | No hotspot | No hotspot | |
| | | | mutation | mutation | mutation | |
| sample 45 | EGFR | p.L858R | 19.50% | No hotspot | No hotspot | Mutation is present in |
| | | | | mutation | mutation | tissues; but no mutation |
| | | | | | | is detected with both |
| | | | | | | methods for cfDNA |
| | TP53 | p.A249K | 13.00% | 1.08% | 1.11% | |
| sample 46 | EGFR | p.H773_V7 | 25.20% | 1.66% | 1.71% | |
| | | 74insPHPH | | | | |
| | TP53 | p.Y234C | 25.30% | 1.63% | 1.68% | |
| sample 47 | TP53 | p.R249S | 48.40% | 0.60% | 0.65% | |
| sample 48 | | | No hotspot | No hotspot | No hotspot | |
| | | | mutation | mutation | mutation | |
| sample 49 | TP53 | p.R158L | 35.80% | 4.44% | 4.31% | |

Note:
No hotspot mutation just means that no hotspot mutation is detected in the region covered by this panel designed by this patent method.

The meanings of the mutation sites in Table 5 above are shown in Table 6:
Table 6 shows the meaning of each mutation site.

| | | |
|---|---|---|
| EGFR | p.L858R | Amino acid of position 858 of the protein encoded by the |
| | | EGFR gene is mutated from L to R |
| TP53 | p.R273L | Amino acid of position 273 of the protein encoded by the |
| | | TP53 gene is mutated from R to L |
| EGFR | p.T790M | Amino acid of position 790 of the protein encoded by the |
| | | EGFR gene is mutated from T to M |
| KRAS | p.G12V | Amino acid of position 12 of the protein encoded by the |
| | | KRAS gene is mutated from G to V |
| EGFR | p.E746_A750del | Amino acid of positions 746 to 750 of the protein encoded |
| | | by the EGFR gene is deleted |
| TP53 | p.R248W | Amino acid of position 248 of the protein encoded by the |
| | | TP53 gene is mutated from R to W |
| TP53 | p.C176F | Amino acid of position 176 of the protein encoded by the |
| | | TP53 gene is mutated from C to F |
| KRAS | p.G12C | Amino acid of position 12 of the protein encoded by the |
| | | KRAS gene is mutated from G to C |
| EGFR | p.A767_V769dup | Amino acids of positions 767 to 769 of the protein encoded |
| | | by the EGFR is repeated |
| PIK3CA | p.E545K | Amino acid of position 545 of the protein encoded by the |
| | | PIK3CA gene is mutated from E to K |
| TP53 | p.V157F | Amino acid of position 157 of the protein encoded by the |
| | | TP53 gene is mutated from V to F |
| EGFR | p.G719S | Amino acid of position 719 of the protein encoded by the |
| | | EGFR gene is mutated from G to S |
| EGFR | p.E709A | Amino acid of position 709 of the protein encoded by the |
| | | EGFR gene is mutated from E to A |
| ALK | p.F1245V | Amino acid of position 1245 of the protein encoded by the |
| | | ALK gene is mutated from F to V |
| TP53 | p.A273C | Amino acid of position 273 of the protein encoded by the |
| | | TP53 gene is mutated from A to C |
| TP53 | p.R249S | Amino acid of position 249 of the protein encoded by the |
| | | TP53 gene is mutated from R to S |
| TP53 | p.Y220C | Amino acid of position 220 of the protein encoded by the |
| | | TP53 gene is mutated from Y to C |
| EGFR | p.L747_T751del | Amino acid of positions 747 to 751 of the protein encoded |
| | | by the EGFR gene is deleted |
| TP53 | p.R337L | Amino acid of position 337 of the protein encoded by the |
| | | TP53 gene is mutated from R to L |
| TP53 | p.H168P | Amino acid of position 168 of the protein encoded by the |
| | | TP53 gene is mutated from H to P |
| EGFR | p.L858R | Amino acid of position 858 of the protein encoded by the |
| | | EGFR gene is mutated from L to R |
| TP53 | p.C176Y | Amino acid of position 176 of the protein encoded by the |
| | | TP53 gene is mutated from C to Y |
| TP53 | p.Y220C | Amino acid of position 220 of the protein encoded by the |
| | | TP53 gene is mutated from Y to C |
| EGFR | p.L747_P753delinsS | Amino acids of positions 747 to 753 of the protein encoded |
| | | by the gene EGFR are deleted, at the same time S is inserted |
| TP53 | p.H179A | Amino acid of position 179 of the protein encoded by the |
| | | TP53 gene is mutated from H to A |
| PIK3CA | p.E542K | Amino acid of position 542 of the protein encoded by the |
| | | PIK3CA gene is mutated from E to K |
| TP53 | p.A248Y | Amino acid of position 248 of the protein encoded by the |
| | | TP53 gene is mutated from A to Y |
| EGFR | p.E746_T751delinsA | Amino acids of positions 746 to 751 of the protein encoded |
| | | by the gene EGFR are deleted, at the same time A is inserted |
| TP53 | p.A249K | Amino acid of position 249 of the protein encoded by the |
| | | TP53 gene is mutated from A to K |
| EGFR | p.H773_V774insPHPH | PHPH is inserted between amino acids of position 773 to |
| | | 774 of the protein encoded by the EGFR |
| TP53 | p.Y234C | Amino acid of position 234 of the protein encoded by the |
| | | TP53 gene is mutated from Y to C |
| TP53 | p.R249S | Amino acid of position 249 of the protein encoded by the |
| | | TP53 gene is mutated from R to S |
| TP53 | p.R158L | Amino acid of position 158 of the protein encoded by the |
| | | TP53 gene is mutated from R to L |

### INDUSTRIAL APPLICATION

The invention has the following advantages due to using the above technical solutions:

1. Easy operation and save time. The conventional capturing and library preparing technology is cumbersome to operate, has a long process, and requires high operator requirements. The invention only involves a one step PCR reaction and corresponding product purification steps, which simplify the operation process and saves time for library preparing (the library preparing can be completed within two hours, and the entire process from the library preparing to the end of the computer sequencing and the completion of the biometric analysis can be completed within 24 hours).

2. Extremely high detection sensitivity. The library preparing method can detect mutations as low as 0.1%. The samples to be tested can be cell free DNA isolated from blood, urine, and CSF, or genomic DNA extracted from conventional frozen tissue, paraffin sections, and fresh puncture tissue.

3. Effectively eliminate cross-contamination between samples. At the beginning of PCR, barcode sequences that distinguish different samples are added, and the simplification of the operation process and steps effectively eliminates cross-contamination that may be caused during the library preparing process.

4. Reduce the cost of the library preparation. Compared to conventional capture techniques, the cost of this library preparation is greatly reduced. The conventional capture probes used for library preparation are costly, and the reagent consumed in their lengthy experimental procedures also adds a lot of cost to the capturing and library preparation. In contrast, the amount of reagents consumed in the one-step library preparation process is greatly reduced. The cost of the library preparing is much lower than the conventional method of capturing and library preparing.

5. Save space. Because this method only requires one PCR reaction, the laboratory requires only three rooms (sample extraction, PCR amplification room, library purification and sequencing). In contrast, 4 rooms (sample extraction, PCR1, PCR2, and library purification and sequencing) are required for conventional library preparing, space requirements is saved.

In addition to detecting tissue samples, the method can quickly, easily, sensitively and specifically target different regions of cell free DNA in samples such as blood, urine, and CSF, and efficiently detect mutations as low as 0.1%. It greatly simplifies the experiment operation, effectively avoids library loss and contamination, significantly reduces costs and improves efficiency.

Flexible and simple library preparing methods and extremely high sensitivity are the biggest features of this patent.

1. A method for preparing an amplicon library for detecting mutation status in regions of target genes of a sample to be tested, comprising the following steps:
1) design and synthesize a Barcode primer F1, an forward primer F2, a reverse outer primer R1, and a reverse inner primer R2,
the Barcode primer F1 consists of a sequencing adapter 1, a barcode sequence for distinguishing different samples, and a common sequence 1 in this order;
the forward primer F2 consists of a common sequence 1, a molecular tag, a specific base sequence, and an forward specific primer sequence in this order;
the reverse outer primer R1 consists of a sequencing adapter 2 and a common sequence 2 in this order;
the reverse inner primer R2 consists of a common sequence 2 and a reverse specific primer sequence in this order;
the sequencing adapter 1 and the sequencing adapter 2 are corresponding sequencing adapters selected according to different sequencing platforms;
the specific base sequence is GAT;
the forward specific primer sequence and the reverse specific primer sequence are primers for amplifying a region to be detected of the target gene;
2) perform a one-step PCR amplification on a ctDNA of the sample to be tested using the Barcode primer F1, the forward primer F2, the reverse outer primer R1 and the reverse inner primer R2, to obtain an amplification product, which is a DNA library used for amplicon sequencing.

2. The method according to claim 1, wherein the barcode sequences are all nucleotides with a length of 8-12 nt, have no continuous the same bases, and the GC content of 40-60%;
or both the common sequence 1 and the common sequence 2 have a length of 16-25 nt, no continuous the same bases, the GC content of 35-65%, and no obvious secondary structure;
or the molecular tag is a sequence having 10-12 random bases;
or the sequencing platform is an Illumina platform, the sequencing adapter 1 is 15, and the sequencing adapter 2 is 17;
or the sequencing platform is an Ion Torrent platform, the sequencing adapter 1 is A, and the sequencing adapter 2 is P.

3. The method according to claim 1, wherein in the PCR amplification, a molar ratio of the Barcode primer F1, the forward primer F2, the reverse outer primer R1 and the reverse inner primer R2 is 6:(10-6):(1-3):(1-3).

4. The method according to claim 1, wherein the mutation is a low-frequency mutation.

5. The method according to claim 1, wherein the sample to be tested is cfDNA isolated from blood of a tumor patient, cfDNA isolated from in vitro urine of a tumor patient, and cfDNA isolated from in vitro CSF or genomic DNA extracted from in vitro tumor tissues of a tumor patient.

6. (canceled)

7. (canceled)

8. (canceled)

9. A method for detecting mutation status in a region to be tested of a target gene of a sample to be tested or for detecting mutation frequencies of mutation sites or mutation regions of target genes in a sample to be tested, comprising the following steps:
1) prepare a DNA library according to the method of claim 1;
2) sequence the DNA library to obtain a sequencing result, and analyze mutation status of a region to be tested of a target gene of the sample to be tested according to the sequencing result.

10. The method according to claim 9, wherein,
the nucleotide sequence of the common sequence 1 is SEQ ID NO: 1 in the sequence listing;
the nucleotide sequence of the common sequence 2 is SEQ ID NO: 2 in the sequence listing;
the nucleotide sequence of the sequencing adapter 1 is SEQ ID NO: 3 in the sequence listing;
the nucleotide sequence of the sequencing adapter 2 is SEQ ID NO: 4 in the sequence listing;
the barcode sequences for distinguishing different samples are SEQ ID NOs: 5 and 14 in the sequence listing, respectively;
the gene to be tested is NRAS, and the corresponding forward specific primer sequence and reverse specific primer sequence are SEQ ID NOs: 15 and 16 or SEQ ID NOs: 17 and 18 in the sequence listing, respectively;
the gene to be tested is ALK, and the corresponding forward specific primer sequence and reverse specific primer sequence are SEQ ID NOs: 19 and or SEQ ID NOs: 21 and 22 or SEQ ID NOs: 23 and 24 or SEQ ID NOs: 25 and 26 or SEQ ID NOs: 27 and 28 or SEQ ID NOs: 29 and 30 or SEQ ID NOs: 31 and 32 in the sequence listing, respectively;
the gene to be tested is PIK3CA, and the corresponding forward specific primer sequence and reverse specific primer sequence are SEQ ID NOs: 33 and 34 or SEQ ID NO: 35 or SEQ ID NO:36 in the sequence listing, respectively;
the gene to be tested is ROS, and the corresponding forward specific primer sequence and reverse specific primer sequence are SEQ ID NOs: 37 and 38 in the sequence listing, respectively;
the gene to be tested is EGFR, and the corresponding forward specific primer sequence and reverse specific primer sequence are SEQ ID NOs: 39 and 40 or SEQ ID NOs: 41 and 42 or SEQ ID NOs: 43 and 44 or SEQ ID NOs: 45 and 46 or SEQ ID NOs: 47 and 48 in the sequence listing, respectively;
the gene to be tested is MET, and the corresponding forward specific primer sequence and reverse specific primer sequence are SEQ ID NOs: 49 and 50 or SEQ ID NOs: 51 and 52 and SEQ ID NOs: 53 and 54 or SEQ ID NOs: 55 and 56 in the sequence listing, respectively;
the gene to be tested is BRAF, and the corresponding forward specific primer sequence and reverse specific primer sequence are SEQ ID NOs: 57 and 58 or SEQ ID NOs: 59 and 60 in the sequence listing, respectively;
the gene to be tested is KRAS, and the corresponding forward specific primer sequence and reverse specific primer sequence are SEQ ID NOs: 61 and 62 or SEQ ID NOs: 63 and 64 in the sequence listing, respectively;
the gene to be tested is TP53, and the corresponding forward specific primer sequence and reverse specific primer sequence are SEQ ID NOs: 65 and 66 or SEQ ID NOs: 67 and 68 or SEQ ID NOs: 69 and 70 or SEQ ID NOs: 71 and 72 or SEQ ID NOs: 73 and 74 or SEQ ID NOs: 75 and 76 in the sequence listing, respectively;
the gene to be tested is ERBB2, and the corresponding forward specific primer sequence and reverse specific primer sequence are SEQ ID NOs: 77 and 78 in the sequence listing, respectively.

11. The method according to claim 9, wherein the sample to be tested is cfDNA isolated from in vitro blood of a tumor patient, and cfDNA isolated from in vitro urine of a tumor patient, cfDNA isolated from in vitro CSF of a tumor patient or genomic DNA extracted from in vitro tumor tissues of a tumor patient.

12. (canceled)

13. (canceled)

14. (canceled)

15. A kit for an amplicon library for detecting mutation status in regions to be detected of target genes of a sample to be tested, comprising a Barcode primer F1, an forward primer F2, and a reverse outer primer R1, reverse inner primer R2 in the method of claim 1.

16. The method according to claim 1, wherein, mutation status comprise mutant bases or amino acids of regions to be detected in target genes of the sample to be tested or the mutation frequencies in regions to be tested of target genes of the sample to be tested.
